(19) United States
(12) Patent Application Publication
(10) Pub. No.: US 2009/0043342 A1
(43) Pub. Date: Feb. 12, 2009

(54) Flat Shaft Fasteners

(76) Inventor: Yosef Freedland, Petah Tikvah (IL)

Correspondence Address: Yosef Freedland, Apt. #2, Nordau 31, Petah Tikvah, 49590, IL

(21) Appl. No.: 12/204,796

(22) Filed: Sep. 4, 2008

Related U.S. Application Data
(63) Continuation-in-part of application No. PCT/IL2007/000377, filed on Mar. 22, 2007.
(60) Provisional application No. 60/786,369, filed on Mar. 28, 2006.
(60) Provisional application No. 60/802,508, filed on May 23, 2006.
(60) Provisional application No. 60/960,338, filed on Sep. 26, 2007.
(60) Provisional application No. 60/996,970, filed on Dec. 13, 2007.

(51) Int. Cl. A61B 17/04

(52) U.S. Cl. 606/313; 606/301; 606/302

(57) ABSTRACT

Disclosed is an expanding fastener. The expanding fastener includes two walls configured to be inserted in a bore in a bone, said two walls include: one first wall having at least one first flat planar wall portion, and one second wall. The expanding fastener further including a shaft having one flat planar shaft portion configured to slide along said one first flat planar wall portion, said shaft further having a shape configured to cause radial outward movement of at least one of said two walls during said sliding.

### RELATED APPLICATIONS

This application is a Continuation in Part of PCT/IL2007/000377, “Curved Wall Fasteners”, filed 22 Mar. 2007, published as WO 2007/110863 which in turn claims priority from:
U.S. Provisional Application 60/786,369, “Expanding Curved Walled Fastener”, filed 24 Mar. 2006; and
U.S. Provisional Application 60/802508, “Compact Tube Fastener”, filed 07 May 2006, the contents of which are incorporated by reference as if fully set forth herein.

This application additionally incorporates by reference as if fully set forth herein:
U.S. Provision Application 60/960,338, “Ram Bolt”, filed 26 Sep. 2007; and
U.S. Provision Application 60/996,970, “Ribbed Fastener”, filed 13 Dec. 2007.

### FIELD AND BACKGROUND OF THE INVENTION

The present invention generally relates to expanding fasteners having at least one flat shaft portion that enters a jacket and causes expansion of the jacket and, more particularly but not exclusively, to biocompatible expanding fasteners for in vivo use.

### Soft Tissue to Bone Fasteners

When soft tissue, for example a ligament or tendon, detaches from the bone, a variety of fasteners may be used to attempt to attach the soft tissue to the bone.

Some fasteners, however, may fail to protect the soft tissue fibers from damage during the attachment procedure, for example from the rotation of screw threads against the soft tissue.

Further, some fasteners may fail to integrate the soft tissue fibers into the bone structure, possibly accruing a weak union between soft tissue and bone, which may tear under low stress.

In screw soft tissue fasteners having heads that protrude out of the bone, the soft tissue is placed under the screw head or under a washer associated therewith, and compressed against the outer cortex of the bone.

Mini anchors anchor in the bone with the uncompressed soft tissue being secured along the bone surface with sutures extending from the anchor.

A mini anchor comprising a wall that surrounds the graft that is compressed by a shaft deployed in a bore in the bone is taught in U.S. Pat. No. 5,268,001 (Nichelson et al), the content of which is incorporated by reference as if fully set forth herein.

An interference screw comprises a headless screw whose threads cut into a graft, is often used to secure an Anterior Cruciate Ligament, (ACL), and/or a Posterior Cruciate Ligament (PCL) to the femur; often possibly accruing the above-note damage to the ligament.

A femoral screw fastener that includes a jacket around the screw threads, to protect the ligament from screw thread damage, is marketed as the BioFix™ by Johnson and Johnson;

A tibial screw fastener that includes a partial jacket around the screw threads is marketed by Cayenne Medical as seen in PCT Application US 2007/006928 (Montgomery et al), now published as WO 2007/109280, the content of which is incorporated by reference as if fully set forth herein.

### Joint Prostheses

Joint prostheses typically have stems that are placed in a bore in the bone to provide support for a prosthetic; and may include a jacket to increase the fastener compression against the bone and/or increase the area of contact between the stem and the bone.

U.S. Patent Applications 2006/0194171; and 2005/0042574 (Lazarof), the content of which are incorporated by reference as if fully set forth herein, teach prosthetic tooth anchors having a jacket that splits to aid in anchoring the prosthetic stem in the bore in the bone.

### Intramedullary Nails

Typical intramedullary (IM) rods with cross pins often require complex technique and instrumentation; an example of an IM rod with cross pins is seen in U.S. Patent Application 2005/0069397 (Shavit, et al), the content of which is incorporated by reference as if fully set forth herein.

### Vertebral Disc Replacement

The bony vertebral bodies of the spine are separated by intervertebral discs, which serve as a cushion that permit controlled motion between vertebral segments. Intervertebral discs degenerate due to trauma, disease, or wear over an extended period, and may compress a spinal nerve that results in leg pain, loss of muscle control, or even paralysis.

In some surgical treatments, an artificial prosthetic disc replacement is interposed between the adjacent vertebrae in place of the degenerated disc, through an anterior approach. An anterior approach requires repositioning of internal organs and may result in inadvertent internal damage during the procedure.

Additional background art includes the following patent, the content of which is hereby incorporated by reference as if fully set forth lo herein:

International Publication Number WO 0197677A2 (Elattrache et al) assigned to Arthex, Inc.

### SUMMARY OF THE INVENTION

According to an aspect of some embodiments of the present invention there is provided an expanding fastener, including: two walls configured to be inserted in a bore in a bone, the two walls including: one first wall having at least one first flat planar wall portion, and one second wall, and a shaft having one flat planar shaft portion configured to slide along the one first flat planar wall portion, the shaft further having a shape configured to cause radial outward movement of at least one of the two walls during the sliding.

According to some embodiments of the invention, at least one of the two walls is configured to secure against at least a portion of the bore in the bone following the radially outward movement.

According to some embodiments of the invention, the one first wall includes a first spine configured to secure a portion of a graft, disposed in the bore, against a portion of the bore.

According to some embodiments of the invention, the graft includes at least one of: a biocompatible material, an autograft, an allograft, and a zenograft.

According to some embodiments of the invention, the first spine includes at least one first rib having a free end extending toward the second wall.

According to some embodiments of the invention, the at least one first rib is configured to press a portion of soft tissue disposed in the bore, against a portion of the bore.

According to some embodiments of the invention, the one second wall includes a second spine.

According to some embodiments of the invention, the second spine includes at least two ribs extending therefrom.

According to some embodiments of the invention, the at least two ribs include: at least one first rib extending from a first side of the second spine, and at least one second rib extending from a second side of the second spine.

According to some embodiments of the invention, prior to the radially outward movement, at least a portion of the one flat planar shaft surface is parallel to the at least one first flat planar wall.

According to some embodiments of the invention, following the radial outward movement, at least a portion the one flat planar shaft surface is parallel to the at least one first flat planar wall.

According to some embodiments of the invention, following the radial outward movement, at least a portion of the one first flat planar wall portion is parallel to the one flat planar shaft portion.

According to some embodiments of the invention, the one first wall includes at least one fold substantially along a longitudinal axis of the fastener.

According to some embodiments of the invention, at least a portion of: the one first wall, and the one second wall, are substantially radially continuous around the shaft.

According to some embodiments of the invention, an elongate stabilizing cord projects from the one first wall, the cord configured to stabilize the position of the one first wall, during the radial outward movement.

According to some embodiments of the invention, an elongate shaft cord projects from the shaft, the cord being configured to pull the shaft during the radial outward movement.

According to another aspect of some embodiments of the present invention there is provided an expanding fastener, including: an elongate shaft, a rearward pointing cone juxtaposed along a rearward portion of the shaft, at least one wall surrounding at least a portion of the elongate shaft, the at least one wall having a rearward edge aligned with a forward surface of the cone, and a cam surface moveably set along a forward portion of the shaft and aligned with a forward edge of the at least one wall, the cam surface configured to cam against, and cause radially outward movement of the at least one wall.

According to an additional aspect of some embodiments of the present invention there is provided an expanding fastener, including: a shaft having two portions, a forward portion and a rearward portion, the rearward shaft portion having a flare, a linearly moveable cam surface slidingly disposed on the forward portion of the shaft, at least one elongate compressed continuous wall member juxtaposed along the shaft and adapted to move radially outward as the cam surface moves toward the flare.

According to a further aspect of some embodiments of the present invention there is provided a method for compressing an in vivo tissue, the method including: boring a bore through a surface of an in vivo tissue, positioning at least two walls in the bore, sliding a shaft between the at least two walls, and causing at least a portion of at least one of the at least two walls to move radially outward and press against at least a portion of the bore.

According to a still further aspect of some embodiments of the present invention there is provided a fastening device, including: a first spine having two sides and spaced a distance from a second spine: the first spine having at least two transverse ribs, at least one rib extending from each of the two sides, toward the second spine, the at least two ribs spaced a distance from each other, and an elongate insertion member insertable into the space for expanding the spines laterally outwardly.

According to another additional aspect of some embodiments of the present invention there is provided a fastening device, including: a first spine spaced a distance from a second spine: the first spine having at least one first transverse rib extending toward the second spine, the second spine having at least one second transverse rib extending toward the first spine and spaced a distance from the at least one first rib, and an elongate insertion member insertable into the space between the first spine and the second spine, thereby expanding the first and second spines laterally outwardly.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings.

With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1F show installation of prior art fasteners;

FIGS. 2A-2B show a perspective and cross sectional view of a wall of a flat shaft fastener, in accordance with embodiments of the present invention;

FIGS. 3A-3B and 4-5 show deployment of a flat shaft fastener, in accordance with embodiments of the present invention;

FIGS. 6-9 show the fastener of FIG. 4B configured for, and being deployed in, a cross section of a knee, in accordance with embodiments of the present invention;

FIG. 10 shows a flat shaft fastener configured for fastening a graft including a bone portion being deployed in a cross section of a knee, in accordance with embodiments of the present invention;

FIGS. 11A-11B show the parts of a flat shaft fastener having dual flanges, in accordance with embodiments of the present invention;

FIGS. 12A-12D show views of parts of a ribbed fastener, according to embodiments of the invention;

FIG. 13 shows an assembled ribbed fastener, according to embodiments of the invention;

FIGS. 14-15 show aerial views of the ribbed fastener of FIG. 13 in unexpanded and expanded configurations, respectively, according to embodiments of the invention;

FIGS. 16-17 and 18A-18B show side and aerial views of a ribbed fastener being deployed in a bone between two ligaments, according to embodiments of the invention;

FIGS. 19-20 show side and aerial views of the ribbed fastener of FIG. 16 being deployed in a bone between two ligaments, according to embodiments of the invention;

FIGS. 21-26 show side and aerial views of the ribbed fastener of FIG. 16 with a graft affixed to the fastener, being deployed in a bone, according to embodiments of the invention;

FIG. 27 shows an exploded view of an alternating ribbed fastener, in accordance with embodiments of the present invention;

FIG. 28 shows parts of the flat shaft fastener of FIG. 11B assembled and being deployed in a spinal portion, in accordance with embodiments of the present invention;

FIG. 29 shows a prior art fastening system deployed in an elbow;

FIG. 30 shows a flat shaft fastener configured for, and being deployed in an elbow, in accordance with embodiments of the present invention;

FIG. 31 shows a flat shaft fastener configured for, and being deployed as a prosthetic stem, in accordance with embodiments of the present invention;

FIG. 32 shows a flat shaft fastener configured for, and being deployed as a fracture fixating Intramedullary, in accordance with embodiments of the present invention;

FIG. 33 shows a flat shaft fastener configured for, and being deployed as a ligament fastener, in accordance with embodiments of the present invention;

FIG. 34A shows a flat shaft fastener having a cross pin, in accordance with embodiments of the present invention;

FIG. 34B shows a flat shaft fastener having a release mechanism, in accordance with embodiments of the present invention;

FIGS. 35A-35C show the flat shaft fastener of FIG. 18A with a key slot and being deployed in a cross section of bone, in accordance with embodiments of the present invention;

FIG. 36 shows the fastener of FIG. 36A having dual shafts and being deployed in a cross section of bone, in accordance with embodiments of the present invention;

FIGS. 37A-37B show the fastener of FIG. 36 having an integral guide wire, in accordance with embodiments of the present invention;

FIG. 37C shows the fastener of FIG. 21B being deployed in a cross section of a knee, in accordance with embodiments of the present invention;

FIGS. 38-40 show exploded and assembled views of a dual flat shaft fastener, in accordance with embodiments of the present invention;

FIG. 41 shows the fastener of FIG. 40 in a cross section of the upper femur, in conjunction with femoral head prosthesis, in accordance with embodiments of the present invention;

FIG. 42 shows a slide nut in cross section, in accordance with embodiments of the present invention;

FIG. 43-44 show the slide nut of FIG. 26 installed in a cross section of the femur, in accordance with embodiments of the present invention;

FIGS. 45-46 show the fastener of FIGS. 21A and 21B configured with a spiral wall and ratchet nut, in accordance with embodiments of the present invention; and

FIGS. 47-48 show side schematic views of embodiments of deployment tools interfacing with fasteners, in accordance with embodiments of the present invention.

### DESCRIPTION OF SPECIFIC EMBODIMENTS OF THE INVENTION

The present invention generally relates to expanding fasteners having at least one flat shaft portion that enters a jacket and causes expansion of the jacket and, more particularly but not exclusively, to biocompatible expanding fasteners for in vivo use.

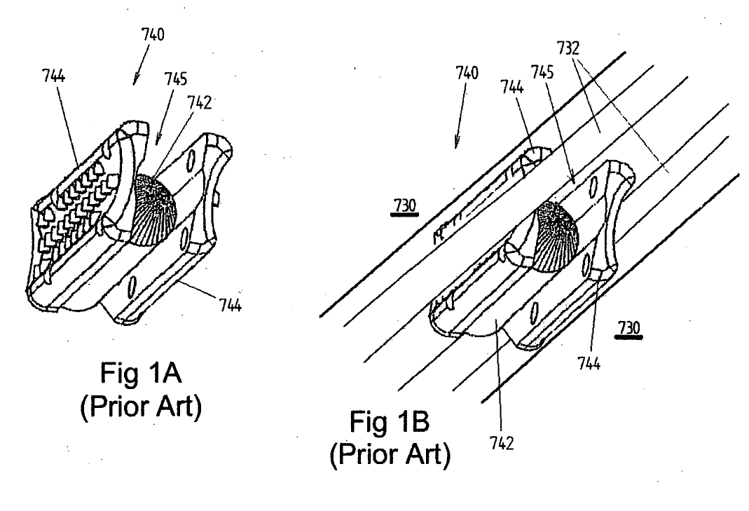

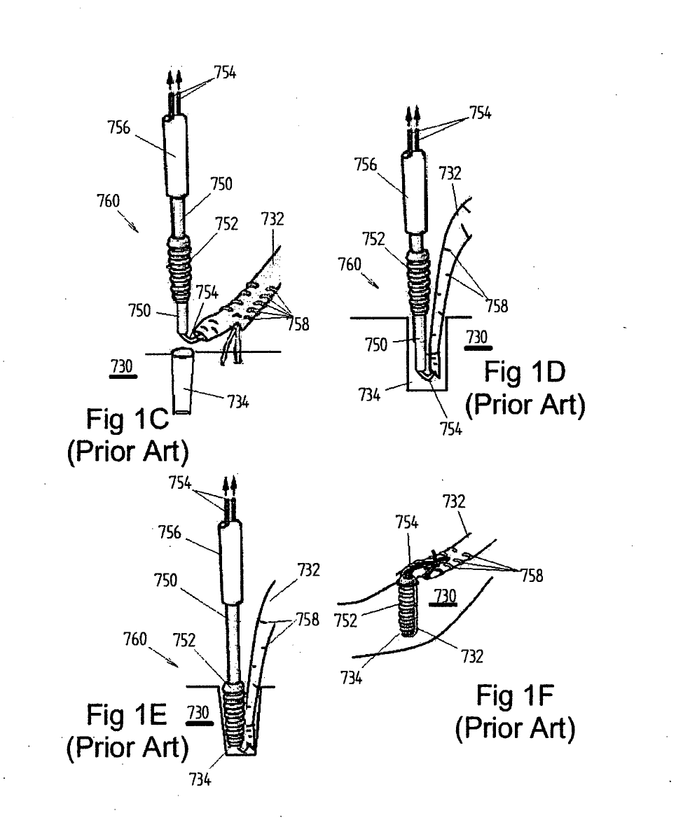

For purposes of better understanding some embodiments of the present invention, reference is first made to existing fasteners as illustrated in FIGS. 1A-1F.

### Existing Fasteners

FIGS. 1A-1B show installation of an existing tibial fastener 740 for securing ligaments to the tibia, marketed by Cayenne Medical, as noted above. Fastener 740 includes a bullet 742 that causes radial outward movement of two curved sheath portions 744; with a resultant space 745 between curved sheath portions 744. Sheath portions 744 compress and secure graft strands 732 against a bone 730 with possible displacement of graft strands 732 between space 745.

FIGS. 1C-1F show installation of an existing fastener system 760 marketed by Arthex for securing tendons to bone, noted above. Fastener system 760 includes a driver 756 slidingly encircling a hollow shaft 750.

To prepare graft strands 732, comprising a tendon, many whip stitches 758 are passed through a long portion of graft strands 732. In addition, robust metal fiber sutures 754 are sutured into graft strands 732 and passed through hollow shaft 750.

By moving hollow shaft 750 downward into a bore 734, as seen in FIG. 1D, graft strands 732 is pulled into bore 734 by metal fiber sutures 754.

Driver 756 is then connected to a screw 752 and rotated to drive screw 752 into bore 734 causing screw 752 to compress graft strands 732 against the walls of bore 734, as seen in FIG. 1E.

Metal fiber sutures 754 may possibly protect graft strands 732 from damage or severance as the threads of screw 752 are rotatingly driven across graft strands 732.

FIG. 1F shows metal fiber sutures 754 tied back around graft strands 732 to provide additional strength to the connection between graft strands 732 and bone 730; possibly creating undesirable bulk.

Before explaining at least one embodiment of the invention in detail, it is to be understood that the invention is not necessarily limited in its application to the details of construction and the arrangement of the components and/or methods set forth in the following description and/or illustrated in the drawings.

The invention is capable of other embodiments or of being practiced or carried out in various ways.

Referring now to the drawings:

### Flat Shaft Fasteners

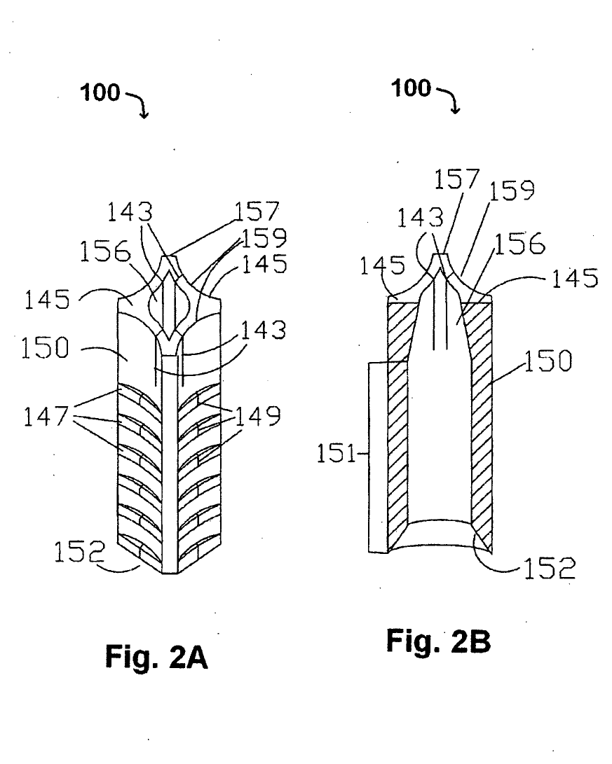

FIGS. 2A and 2B show portions of a flat shaft fastener 100 comprising perspective and cross sectional views of a sheath 150 having an axial bore 156 and a cam edge 152. Sheath 150 is shown in a pre-deployed configuration and, optionally, formed into a four sided curvilinear configuration including walls 159 that join at folds 157.

While flat shaft fastener 100 comprises a four-sided polygon shape, other non-limiting shapes of sheath 150 are contemplated, including a hexagon transverse cross section shape; the shape and configuration of sheath 150 being well-known to those familiar with the art.

While sheath 150 is shown with four contiguous walls, in embodiments, sheath 150 can comprise two wall portions, each consisting of two joined walls 159 similar to that shown in FIG. 2B.

Alternatively, each side of sheath may have a “c” shaped cross section, as will be explained below.

While fold 157 is shown substantially parallel to a longitudinal axis of flat shaft fastener 100, fold 157 is optionally positioned anywhere between zero degrees and 60 degrees to the longitudinal axis of flat shaft fastener 100.

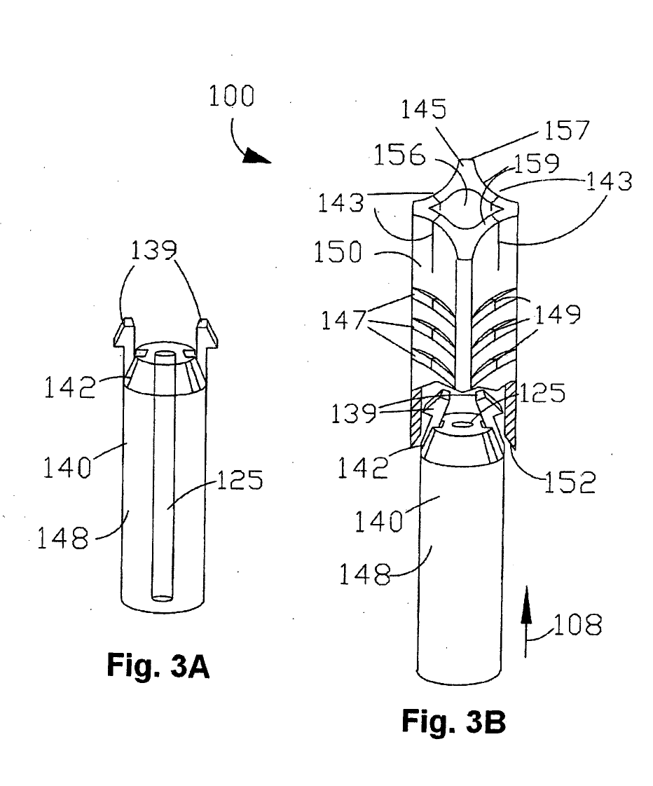

A shaft 140 having an axial guide channel 125 and securing tabs 139 is shown in FIG. 3A. FIG. 3B shows a cross sectional view of sheath 150 comprising upper slits 143 and a thick upper portion 145; thick upper portion 145 moving radially outward during deployment, as will be explained below.

To deploy flat shaft fastener 100, securing tabs 139 are compressed radially inward and shaft 140 is pressed in an upward direction 108 so that a shaft cam surface 142 cams against cam edge 152.

Continued pressure from cam surface 142 on cam edge 152 causes axial bore 156 to expand and thereby allow a shaft support surface 148 to enter axial bore 156.

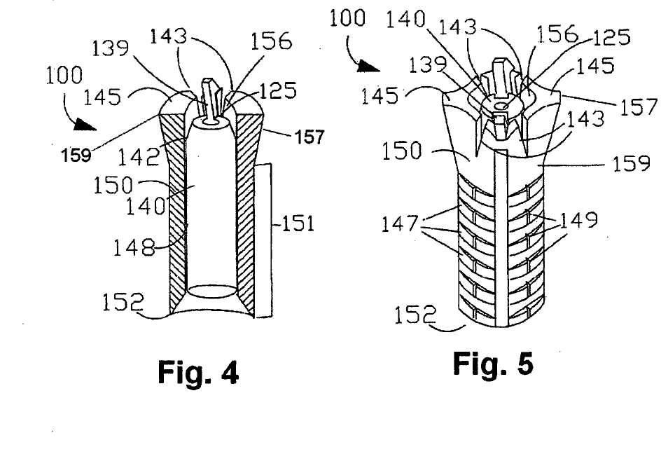

FIG. 4 shows a cross section of shaft 140 within axial bore 156 wherein fold 157 has unfolded and walls 159 have moved radially outward to form a substantially tubular round configuration.

Additionally, slits 143 have opened while thick upper portions 145 have been pushed radially outward by the pressure from shaft 140.

As seen in FIG. 5, projection slits 149 have opened, allowing outward projections 147 to assume a substantially circular configuration.

In the embodiment shown, rows of outward projections 147 comprise ribs. In other embodiments, outward projections 147 comprise rows of multiple outwardly horizontally disposed bars or any one of many other projection shapes.

In the embodiment shown, rows of outward projections 147 are substantially perpendicular to the longitudinal axis of the fastener. In other embodiments, rows of outward projections 147 are between 90 degrees and 170 degrees to longitudinal axis of the fastener; the many configurations and angles of outward projections 147 being well-known to those familiar with the art.

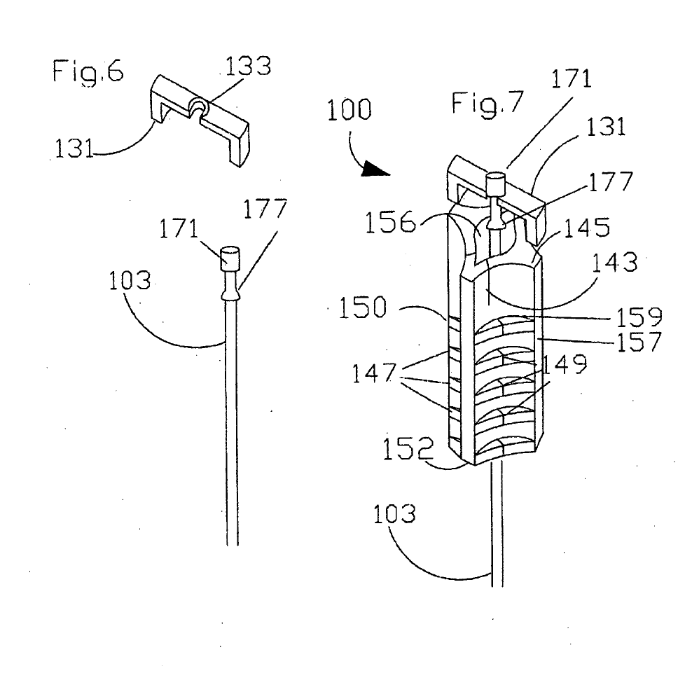

The in vivo deployment of flat shaft fastener 100 may be facilitated with one or more ancillary devices. Referring to FIGS. 6-7, flat shaft fastener 100 optionally includes a guide wire 103 having a transverse bar 131. Transverse bar 131 optionally includes a slot 133 that slides around guide wire 103, to position transverse bar on a support shelf 177 under a retaining head 171.

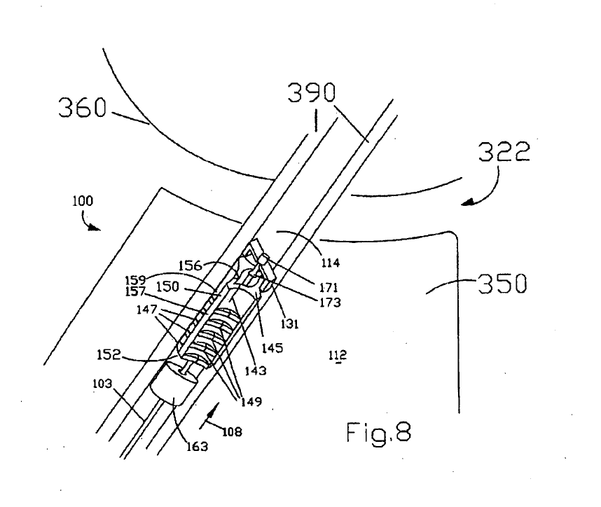

FIG. 8 shows a cross section of a knee joint 322. Graft strands 390 extend from a femur 360 and are pressed radially outward against a portion of a trabecular bone 112 within a tibia 350. Flat shaft fastener 100 is typically inserted in the collapsed configuration between graft strands 390.

As used herein, the terms proximal and proximally refer to positions and movement, respectively, deeper into bore 114, and/or toward femur 360. As used herein, the terms distal and distally refer to positions and movement respectively outward, or away, from bore 114; and/or into femur 360.

In embodiments, graft strands 390 comprise an autograft from the pes anserinus of the leg. An alternative source of graft strands 390 include grafts grown ex vivo from stem cells is taught by Altman, et al, in U.S. Pat. No. 6,217,340, the disclosure of which is incorporated herein in its entirety by reference

Alternatively, graft strands 390 comprise soft tissue harvested and prepared from a cadaver.

In still further embodiments, graft strands 390 may be reinforced with a variety of fibers from man-made materials, including biologically compatible polymers and/or matrixes that aid in fostering graft strength post-operatively.

An example of an artificial graft enhancer is marketed as the LARS artificial ligament (Ligament Advanced Reinforcement System) by Surgical Implants and Devices, Arc-sur-Tille, France); the many sources for graft strands 390 being well-known to those familiar with the art.

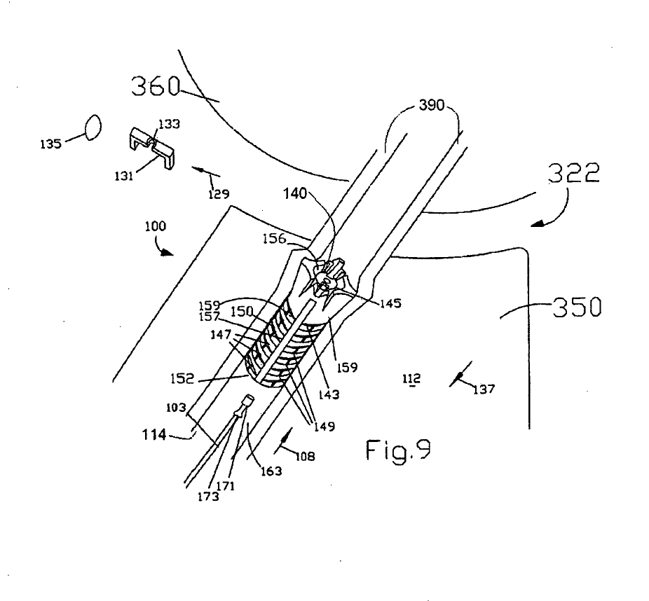

As seen in FIG. 9, following radially outward movement, transverse bar 131 is freed of guide wire 103 and pulled in a side direction 129. When deployed in arthroscopic procedures, transverse bar 131 is optionally accessed through a side portal 135; using instrumentation that is well known to those familiar with the art.

Following removal of transverse bar 131, guide wire 103 is pulled out of shaft 140 in a downward and distal direction 137.

Sheath 150 and/or shaft 140 do not include projections to secure sheath 150 against rotation during deployment, as is the case with the above-noted Biofix by JnJ.

Without such projections, flat shaft fastener 100 is optionally fully contained within bore 114. The inventor has discovered that maintaining entire flat shaft fastener 100 in bore 114 may possibly reduce irritation by flat shaft fastener 100 on the thin skin covering the anterior tibia, the location of the JnJ Biofix projection.

Sheath 150 is compressed against graft strands 390 substantially without forward or rearward translation of sheath 150, so there is possibly minimal shear force that is transferred to graft strands 390 during outward movement, thereby possibly minimizing damage to graft strands 390, alternatively referred to as “preserving integrity of graft strands 390.

Additionally or alternatively, sheath 150 is compressed against graft strands 390 without rotational translation of sheath 150, so there is possibly minimal cutting action of outward projections 147 into graft strands 390 during outward movement, as may be the case with unsheathed kurosaka screws.

While flat shaft fastener 100 has been shown in securing graft strands 390 in knee 322, flat shaft fastener 100 may be adapted for securing a variety of soft tissue portions of varied thicknesses to any bone in the body. Just a few of the many embodiments and uses for flat shaft fastener 100 are now presented.

### Bone Tendon Bone Grafts

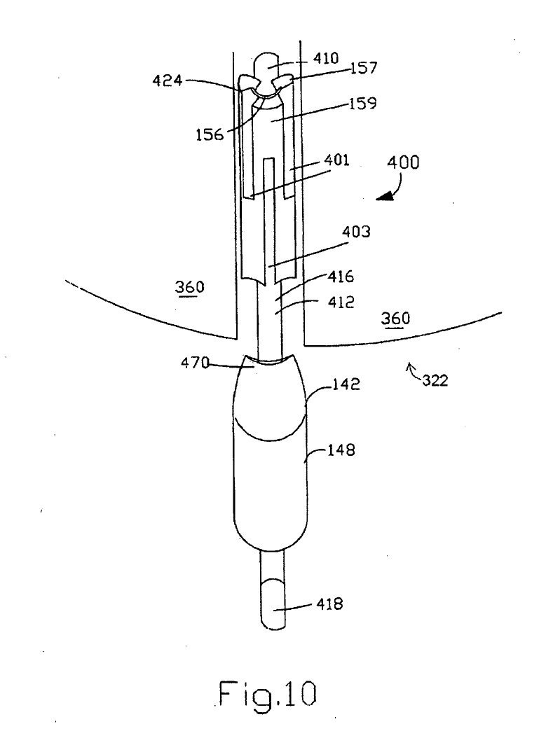

FIG. 10 shows a graft fastener 400 configured for use with a bone tendon bone graft 416. Bone tendon bone graft 416 comprises a first bone portion 410 that installs in a femur 360; a second bone portion that installs in the tibia (not shown); and a ligament portion 412 that spans between femur 360 and the tibia.

In embodiments, a sheath 424 upper end is formed into an arc-shaped transverse cross section that partially encircles at least a portion of bone portion 410. An upper cam portion 470 is similarly shaped in the transverse cross-section in an arc to facilitate shaft 142 sliding into axial bore 156 during expansion of graft fastener 400.

In embodiments, sheath 424 includes wide slots 401 that extend downward from the upper portion of graft fastener 400 and wide slots 403 that extend upward from the lower portion of graft fastener 400.

Wide slots 403 pass in between, and optionally parallel to, wide slots 401 in the center of sheath 424.

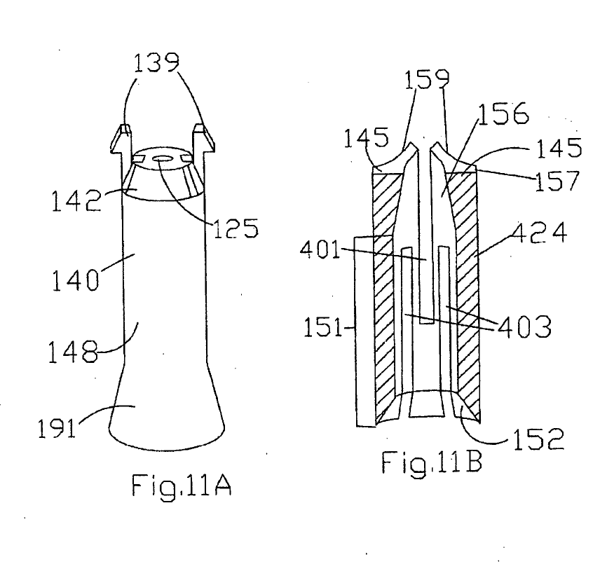

In embodiments seen in FIGS. 11A-11B, flat shaft 140 optionally includes a conical lower portion 191 that causes lower wide slots 403 to separate, so that during expansion the lower portion of sheath 424 moves radially outward with respect to the middle portion of sheath 424. Lower wide slots 403, in conjunction with conical lower portion 191, allow sheath 424 to transfer significant radial pressure against bone portion 410.

### Ribbed Fastener

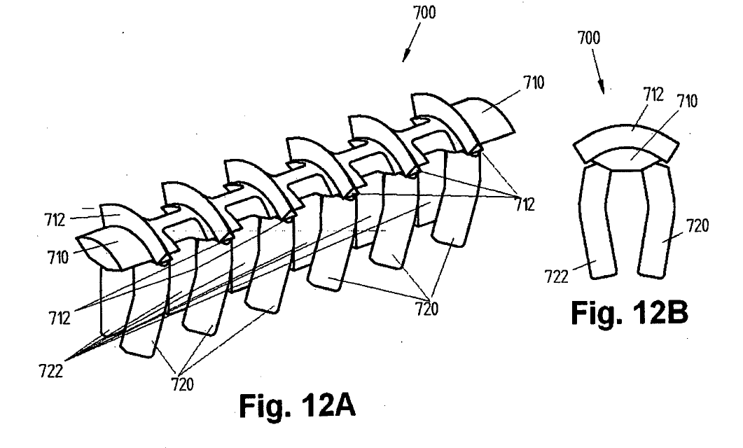

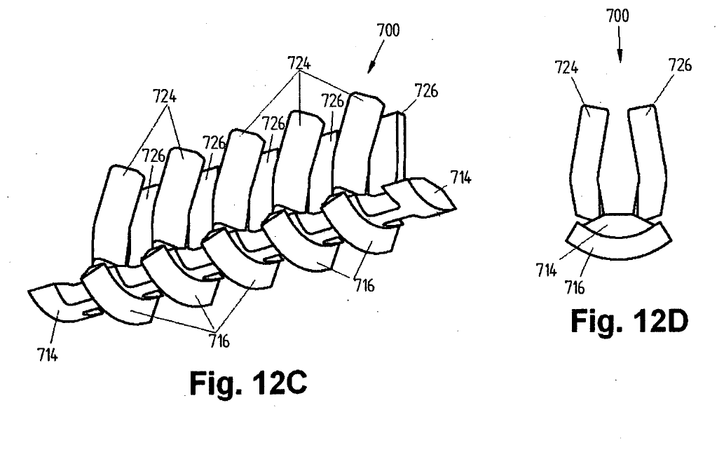

FIGS. 12A-12B show a side view and aerial view respectively of portions of a ribbed fastener 700 which comprises a first spine 710 with a central projection 712 transversely projecting therefrom; and ribs 720 and 722 projecting from either side of first spine 710.

FIGS. 12C-12D show a side view and aerial view respectively of ribbed fastener 700 with a central projection 716 transversely projecting therefrom and ribs 724 and 726 projecting from either side of a second spine 714.

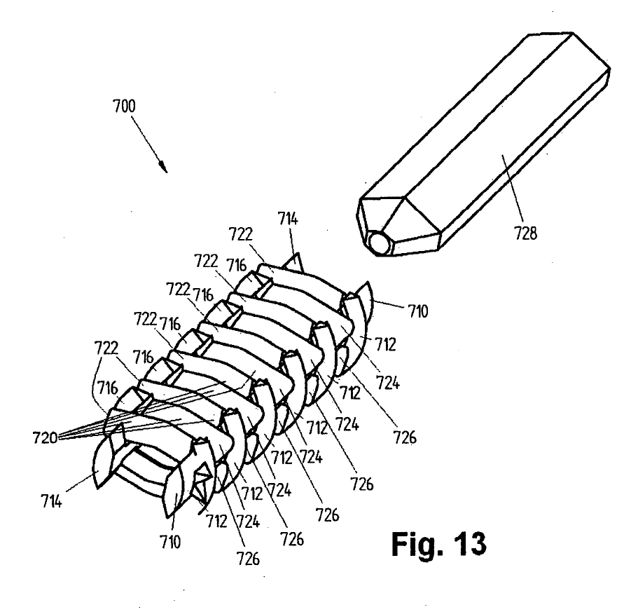

FIG. 13 shows assembled ribbed fastener 700 in which ribs 720 project toward second spine 714, between ribs 724. Ribs 724 similarly project toward first spine 710 between ribs 720.

Additionally shown is a shaft 728 which is pressed into the space between spines 710 and 714 during deployment of ribbed fastener 700.

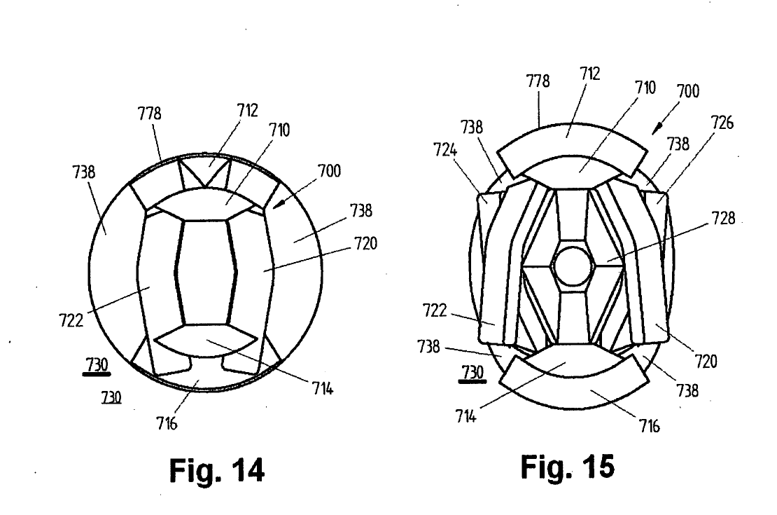

FIGS. 14-15 show aerial views of ribbed fastener 700 in a bore 738 in expanded and unexpanded views respectively.

As shaft 728 is pressed into the space between spines 710 and 714, spines 710 and 714 are expanded radially outward to secure in bone 730.

As seen in FIG. 15, optionally, ribs 720 and 722 are flexible and/or flexibly connected to spines 710 and 714, respectively, and swing radially outward from spines 710 and 714 to secure in bone 730.

Further, in optional embodiments, shaft 728 and ribs 726 and 722 are similarly flexible and swing radially outward to secure in bone 730.

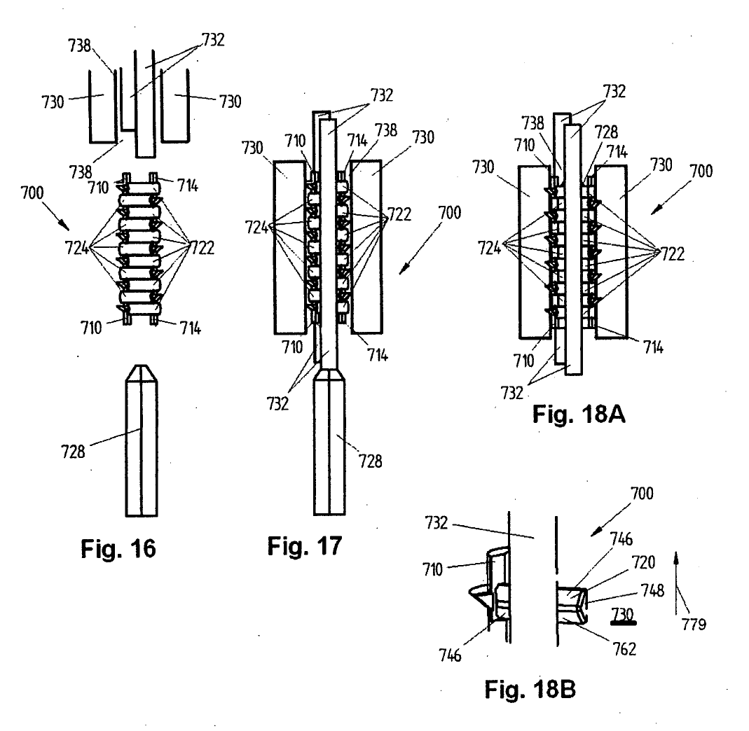

FIGS. 16-18A show schematic views of ribbed fastener 700 being pressed into bore 738 between graft strands 732.

Graft strands 732 may for example, comprise a ligament graft for repairing an ACL in bore 738 extending through the tibia.

Alternatively, graft strands 732 may comprises severed portions of a tendon that must be reattached to the bone 730, a procedure referred to tenodesis. Examples of tenodesis include reattachment of a severed head of the biceps to the radius and reattachment of finger tendons to the digital bones.

Embodiments of ribbed fastener 700 may additionally be used in aligning and securing bone fractures; the many applications of fastener 700 being well known to those familiar with the art.

FIG. 18B shows details of rib 720 which optionally comprises a projection 762 having a lower edge 746 configured to aid in preventing migration of graft strands 732 in a direction 779 against forces that typically pull on graft strands 732.

There are many alternative configurations for rib 720 that aid in preventing migration of graft strands 732 and/or aid in securing to bone 730 that may be contemplated in configurations of ribbed fastener 700; and are known to those familiar with the art.

For example, to prevent damage to particularly thin graft strands 732, the angle between lower edge 746 and an upper edge 746 may be increased.

Rib 720 may additionally include a blunt edge 748 for securing particularly delicate graft strands 732 or soft tissue, while possibly minimizing any damage to the graft strands 732.

Additionally, ribs 720, 722, 724 and 726, may be manufactured relatively thick, or alternatively relatively thin with relatively small intervening spaces therebetween; the latter, for example; to aid in fastening against hard bone 730.

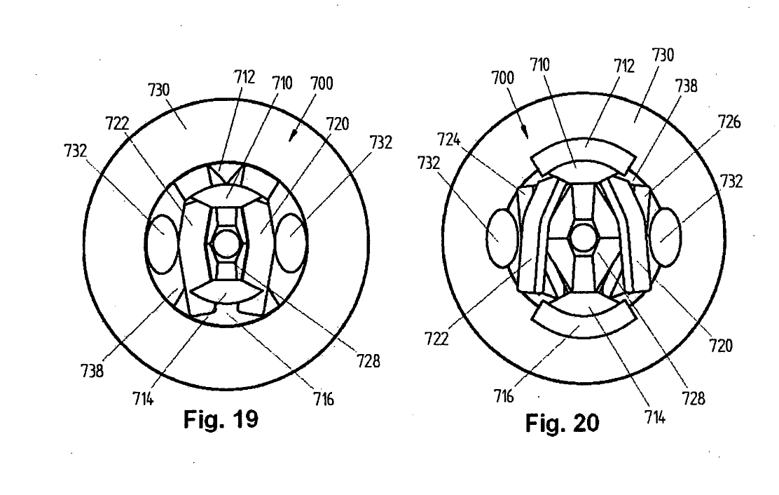

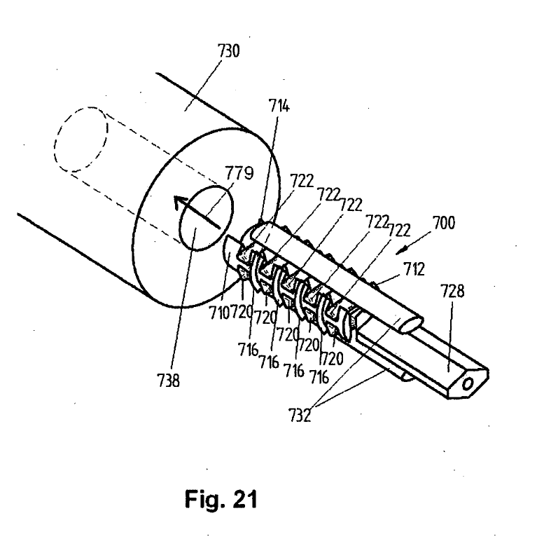

FIGS. 19-20 show unexpanded and expanded aerial views of ribbed fastener 700 in bore 738. As shown in FIG. 20, upon expansion of ribbed fastener 700, ribs 720, 722, 724 and 726, optionally expand radially outward to cause graft strands 732 to deform bone 730 around bore 738. Such might occur when graft strands 732 comprise an ACL soft tissue graft and bone portion 730 comprises soft cancellous portions of the tibia.

### Tendon Attachment

FIG. 21 shows a length of graft strands 732 forming a curve between spine 710 and spine 714; a configuration that might be used in tenodesis.

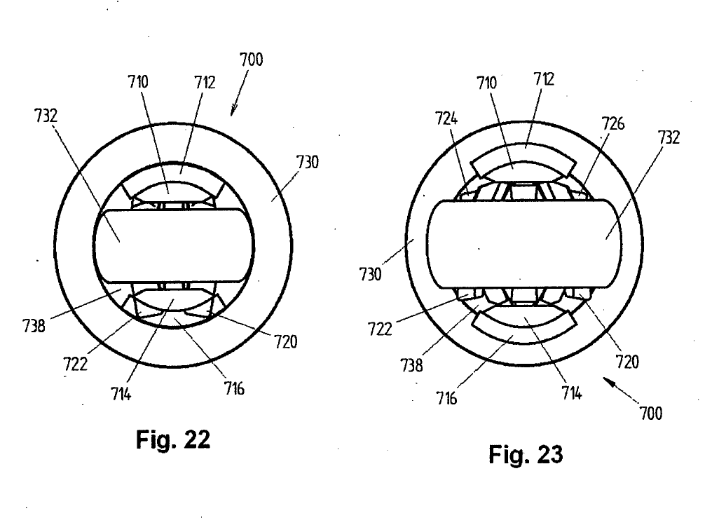

FIGS. 22-23 show ribbed fastener 700 in unexpanded and expanded aerial views, respectively, demonstrating that graft strands 732 pass between spines 710 and 714 and continue on either side of ribbed fastener 700.

In embodiments, spines 710 and 714 optionally extend a distance above ribs 720, 722, 724 and 726 to serve as guide posts to maintain the position of graft strands 732.

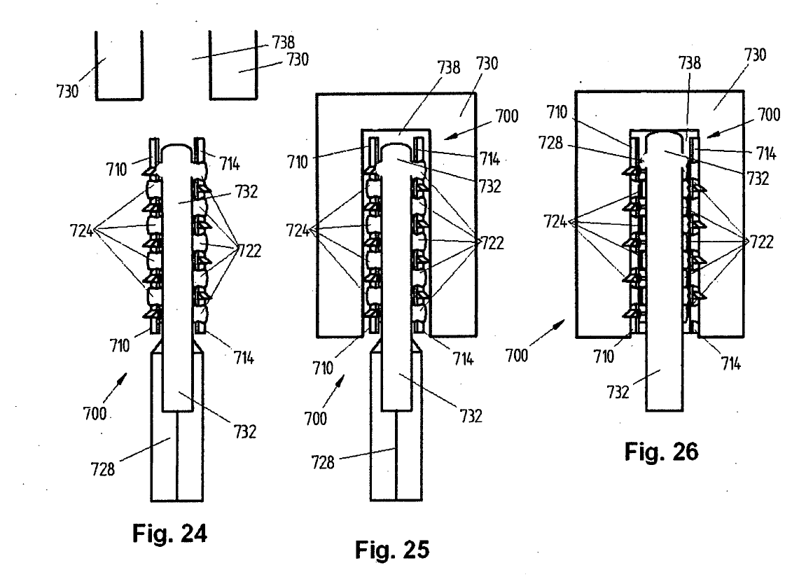

FIGS. 24-26 show schematic views of ribbed fastener 700 and graft strands 732 being pressed into bore 738.

There are many embodiments of ribbed fastener 700, the following embodiment being just one such example.

### Alternating Ribbed Fastener

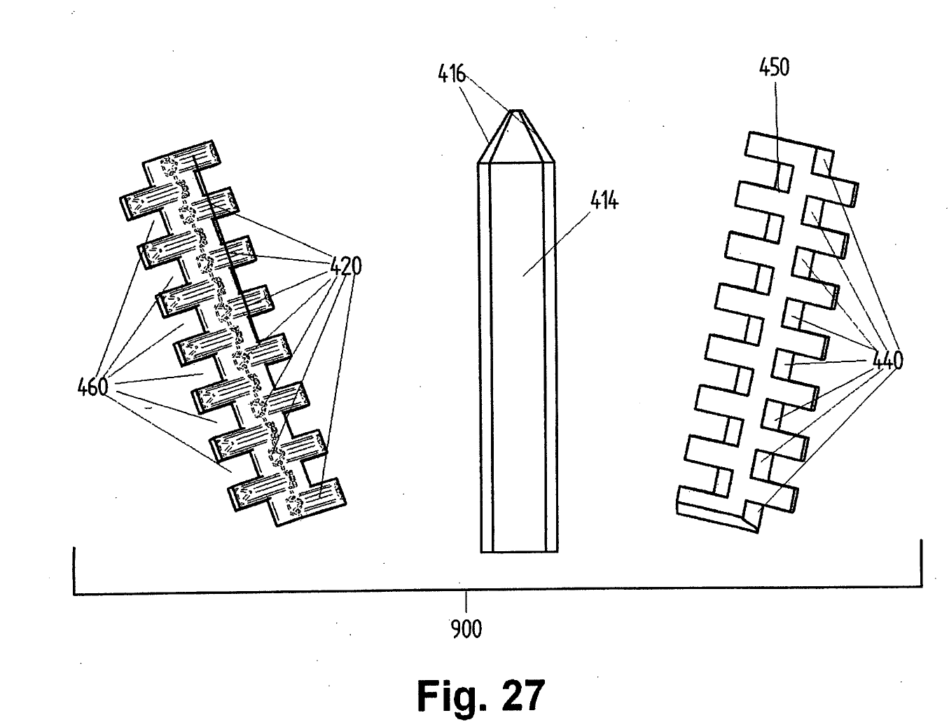

FIG. 27 shows an exploded view of an alternating ribbed fastener 900, in which spaces 460 are interposed between ribs 420 that alternate from side to side of a rib 450 extending from a beveled shaft 440; a configuration that may possibly aid in holding soft tissue in a bone bore. Alternating ribbed fastener 900 additionally includes a deployment shaft comprising an elongate section 414. The shaft further includes an upper tapered section 416 that aids in interposing between beveled shafts 440.

Dynamic spinal stabilizers have shown potential in alleviating back pain. However, some are of a large size that requires a large surgical incision; with the possibility of known sequella. The following is just one example of a dynamic spinal stabilizing fastener according to embodiments of the invention.

### Dynamic Spinal Stabilizer

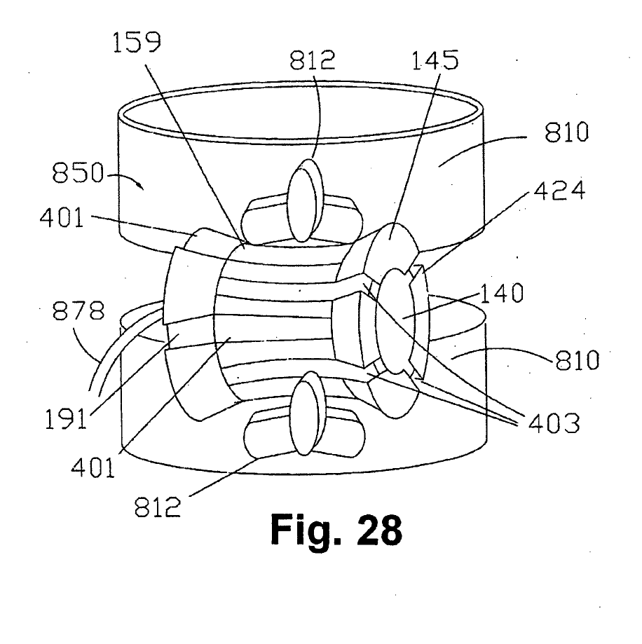

FIG. 28 shows a dynamic spinal stabilizing fastener 850 including shaft 140 and sheath 424 assembled between spinal vertebrae 810. Dynamic spinal stabilizing fastener 850 is configured for providing substantial bulk to separate vertebral processes 812, thereby separating vertebral bodies 810 and removing pressure from a nerve root 878.

Sheath 424 includes lower slots 401 and upper slots 403 that open to form oblique portions which stabilize fastener 850 on either side of posterior processes 812.

Dynamic spinal stabilizing fastener 850 inserts through a small incision with minimal dissection in a compact configuration and then radially enlarges to separate vertebral processes 812.

### Existing Elbow Fracture Repair

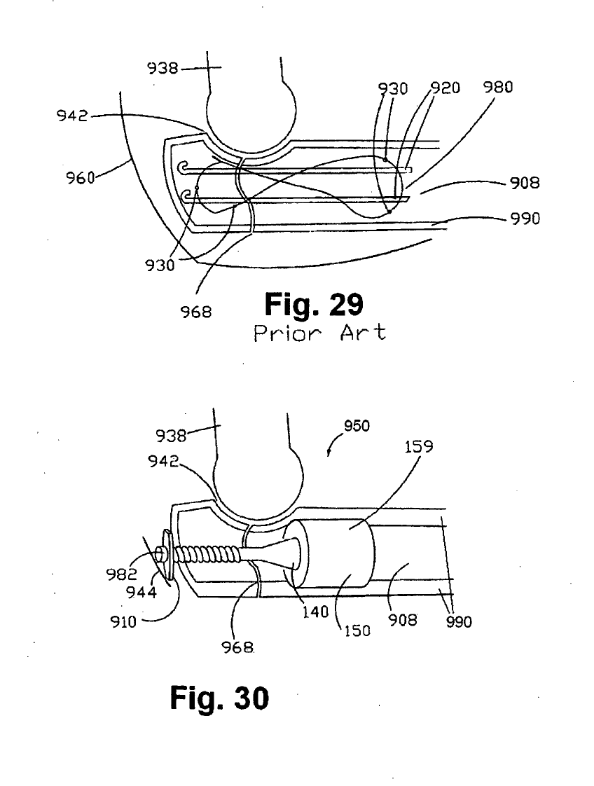

FIG. 29 shows an existing technique for “open reduction internal fixation” (ORIF) of an elbow fracture 968 in a radius bone 908, just below a humerus 938. The center of radius 908 is soft and does not support screws (not shown). Installing screws through a cortex 990 on either side of fracture 968 can cause damage in a joint space 942.

To provide proper fixation without damage to joint space 942, Kirschner wires 920 are driven across fracture 968. A monofilament wire 980 is then passed through drill holes 930 in cortex 990 and formed into a figure eight that provides compression on fracture 968.

A major drawback of this prior art procedure is that considerable dissection is required to accommodate Kirschner wires 920 and monofilament wire 980; typically a “hockey stick” incision 960, perhaps 17 to 22 centimeters in length. Incision 960, unfortunately will likely heal with considerable scarring, impeding range of motion (ROM) of joint 948.

### Flat Shaft Elbow Fastener

In distinct contrast, as shown in FIG. 30, a flat shaft elbow fastener 950 installs through a relatively small incision 944, perhaps two centimeters in length, resulting in a small scar that will likely not impede elbow ROM.

Sheath 150 is radially enlarged on one side of fracture 968 and secures against a wide area of bone between cortex 990 walls.

Shaft 140 passes through fracture 968 and is fitted with a nut 982 and a washer 910 that adjustably compress cortex 990, thereby compressing fracture 968.

### Prosthetic Stem

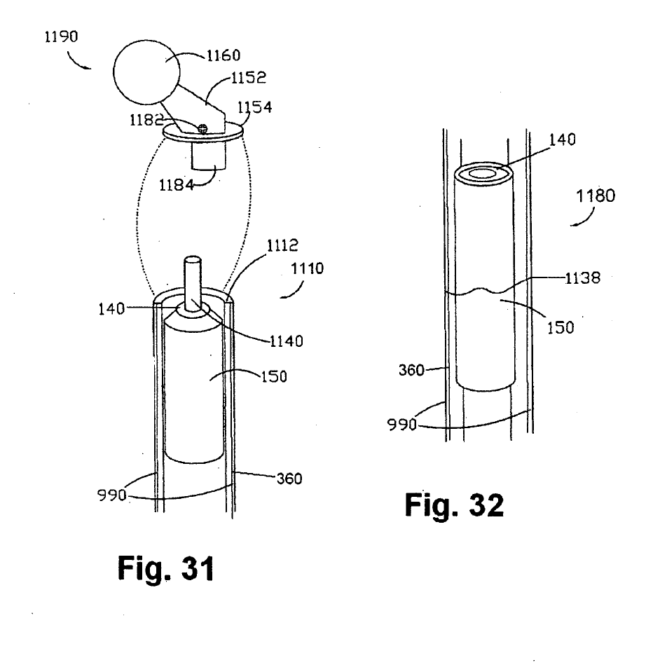

FIG. 31 shows a prosthetic stem fastener 1110 that supports a femoral prosthetic head 1190. Femoral prosthetic 1190 includes: a femoral head 1160; a femoral neck 1152; a prosthetic platform 1154 that rests upon a cortical edge 1112; and a nut 1182 that is rotated to secure a prosthetic stem 1184 on a post 1140 which is an extension of shaft 140.

Sheath 150 is compressed radially outward against cortex 990; thereby spreading forces generated at femoral prosthetic head 1160 to a large surface area of cortex 990; which in turn helps maintain integrity of cortex 990 against the damage caused by focal pressures of an existing prosthetic stem.

### Intramedullary Rod

FIG. 32 shows an IM fracture fastener 1180 fastening anchored against cortex 990 while stabilizing and aligning a fracture 1138. Sheath 150 contacts a large area of bone 360, thereby possibly providing optimal stabilization of fracture 1138 and/or preventing torque.

### Mini Anchor

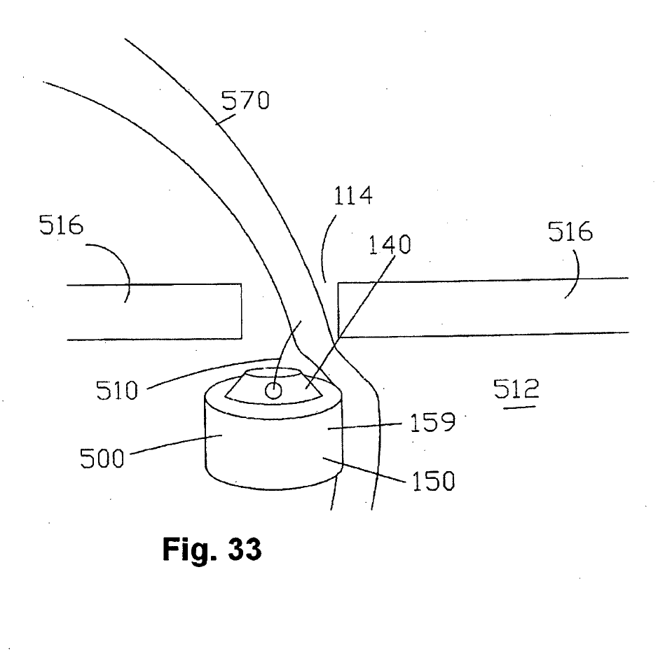

FIG. 33 shows a mini anchor fastener 500 for securing soft tissue 570 in a trabecular bone 512. Mini anchor fastener 500 is used, for example, when soft tissue 570 comprises a ligament that has ripped away from an attachment to a bone cortex 516.

In embodiments, mini anchor fastener 500 in the pre-deployed configuration is configured to have a cross sectional area that is less than the cross sectional area of bore 114, thereby allowing placement of soft tissue 570 in bore 114 along with fastener 500.

To secure soft tissue 570, bore 114 is made through cortex 516 and optionally into trabecular bone 512. Fastener 500 is introduced into bore 114, along with soft tissue portion 570 so that a portion of soft tissue 570 passes through a portion of cortex 516.

Mini anchor fastener 500 is expanded, optionally below a portion of cortex 516, thereby compressing soft tissue 570 against trabecular bone 512 where there are appropriate vascular channels to supply healing-promoting nutrients to soft tissue 570. The radially enlarged mini anchor fastener 500 additionally compresses and secures the position of soft tissue 570.

Following compression of soft tissue 570, a suture 510, is optionally passed through soft tissue 570 and tied, thereby providing further stability of soft tissue 570.

The inventors have found that introducing soft tissue 570 along with fastener into trabecular bone 512, may possibly allow easier positioning of soft tissue 570 than that provided by the above-noted Arthrex Biotenodesis™.

### Alternative Configurations

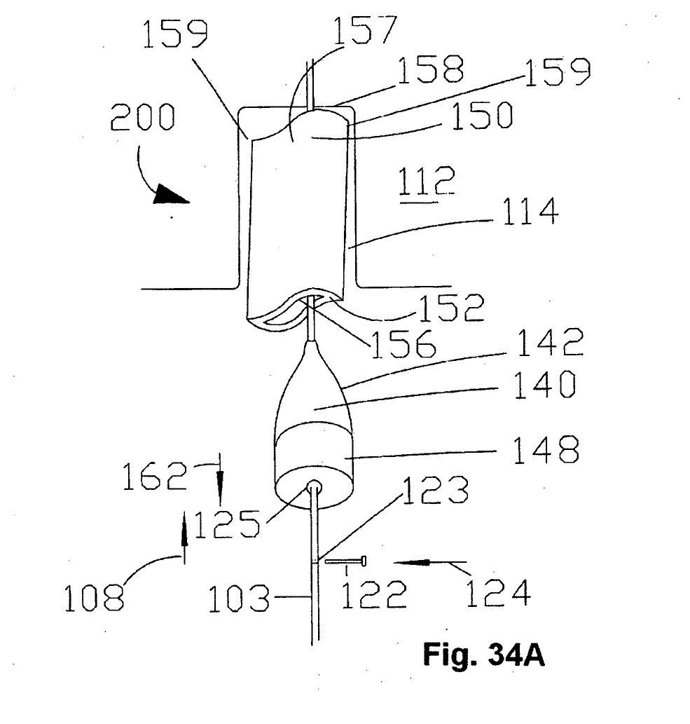

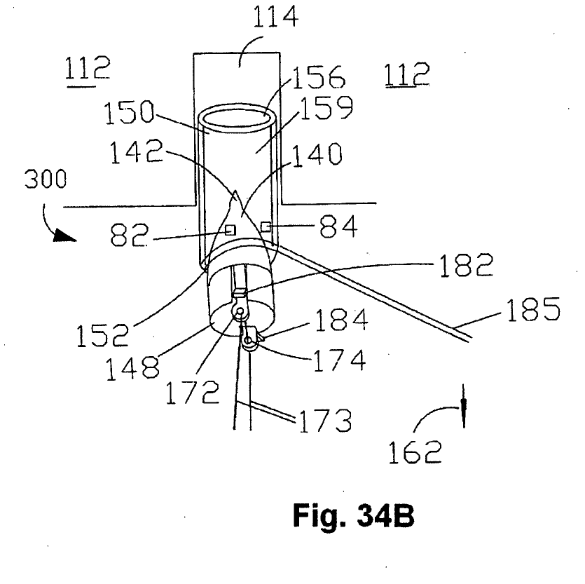

FIG. 34A shows a sheathed fastener 200 comprising a sheath 150 in a pre-deployed configuration with respect to bore 114 in trabecular bone 112.

As used herein, the term sheath 150 refers to a sleeve, jacket or tube having an axial space or bore into which shaft 140 is deployed.

To allow sheath 150 to be easily placed in bore 114 in the pre-deployed configuration, sheath 150 is optionally formed into a flat curvilinear configuration, or alternatively a flat planar cross sectional configuration, allowing the originally compact circumference to reach maximum diameter upon expansion.

Alternatively, sheath 150 may comprise two curvilinear flat walls, or two flat, substantially planar flat walls, that expand radially outward.

In embodiments, guide wire 103 includes a transverse pin 122 that slides in a direction 124 into a transverse pinhole 123. While transverse pin 122 is shown being slidingly attached to pin hole 123, pin 122 may be fixed to guide wire 103 and comprise any one of several radially outwardly projecting configurations.

To secure sheathed fastener 200 in bore 114, guide element 103 is pressed in an upward direction 108 so transverse pin 122 presses shaft 140 upward. Shaft 140 movement causes the upper portion of sheath 150 to press and stabilize against an upper surface 158 within in vivo tissue bore 114. With continued pressure, sheathed fastener 200 enlarges within bore 114 to secure tissue in bore 114.

Following radial outward movement of sheathed fastener 200, guide wire 103 is removed from guide channel 125, for example by pulling guide wire 103 in a distal direction 162. Alternatively, transverse pin 122 is pulled out of guide wire 103 and guide wire 103 is pulled out of shaft 140 in proximal direction 108.

FIG. 34B shows a sheathed fastener 300 including eyelet legs 172 and 174 having tines 182 and 184, respectively; and being held by a grasping instrument 185.

Upon positioning sheath 150 around shaft support surface 148, tines 182 and 184 lock into flat shaft tine receptacles 82 and 84 respectively, preventing movement of shaft 140 in sheath 150.

To remove shaft 140 from sheath 150, a tine cord 173 is pulled in direction 162 to cause legs 172 and 174 to move toward each other and release tines 182 and 184 from tine receptacles 82 and 84.

Alternatively, legs 172 and 174 which extend beyond sheath 150 when shaft 140 is contained within sheath 150, are pressed toward each other using an instrument, for example a forceps (not shown), to facilitate removal of shaft 140 from bore 156.

Legs 172 and 174 allow an operator to remove sheathed fastener 300, for example for repositioning sheath 150 and or retensioning graft strands 390 shown above. As the interface between surface of sheath 150 and bore 114 is non-threaded, removal of sheathed fastener 300, adjustment of sheathed fastener 300 position, and further deployment of sheathed fastener 300, is optionally repeated without compromising the integrity of trabecular bone 112.

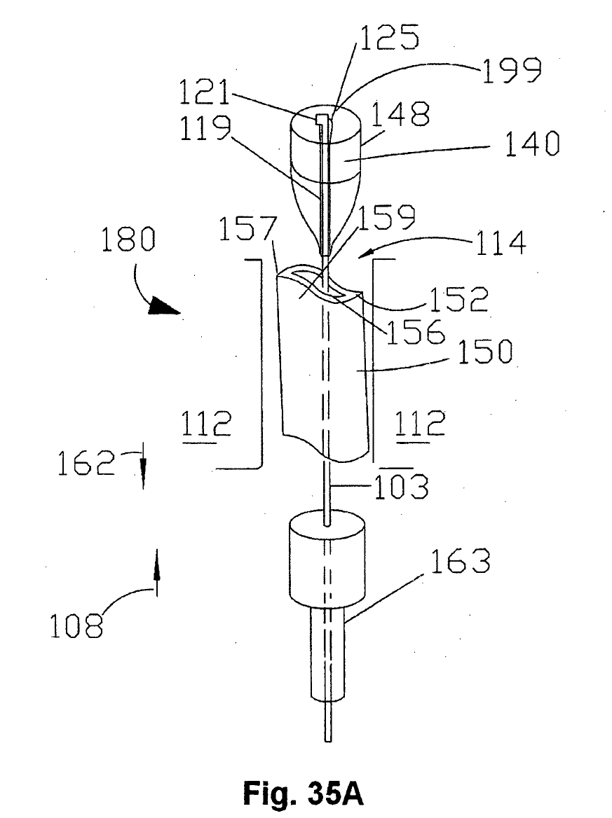

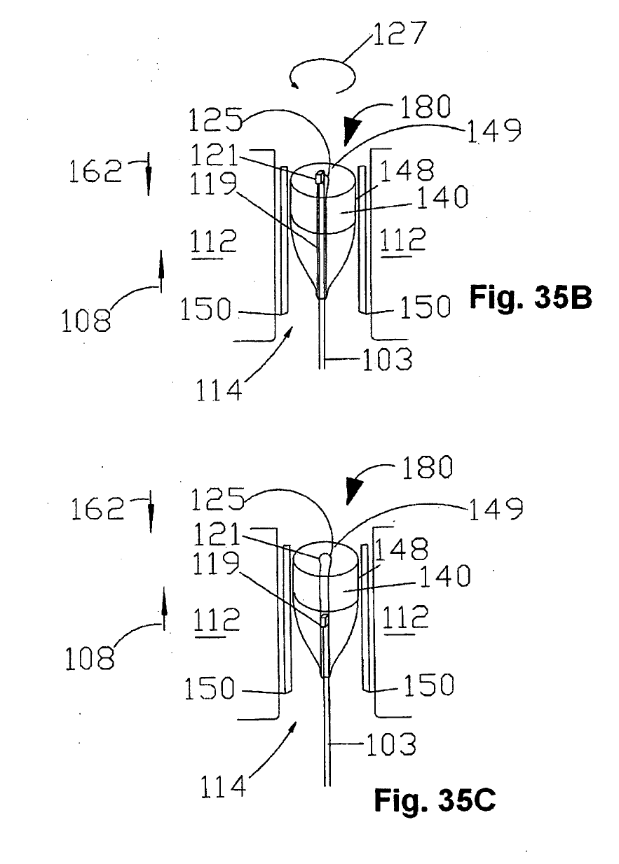

FIG. 35A shows a flat shaft fastener 180 in which shaft 140 has a longitudinal channel 119 and guide wire 103 has a radially outward extending end projection 121.

Sheath 150 is positioned within bore 114. Guide wire 103 is pulled in distal direction 162 to cause end projection 121 to press on a shaft upper surface 199. A tool 163 is used to stabilize sheath 150 and shaft 140 is pulled into sleeve 150 as end projection 121 is pulled in distal direction 162.

Following shaft 140 being pulled into sheath 150, as seen in FIG. 35B; guide wire 103 is rotated in a direction 127 to align end projection 121 with key channel 119. As seen in FIG. 35C, guide wire 103 is pulled downward in direction 162. End projection 121 slides through key channel 119 and guide wire 103 is pulled out of shaft 140. Fastener embodiment 180 optionally dispenses with the need to remove pin 122 separately from guide wire 103; as shown in FIG. 34A.

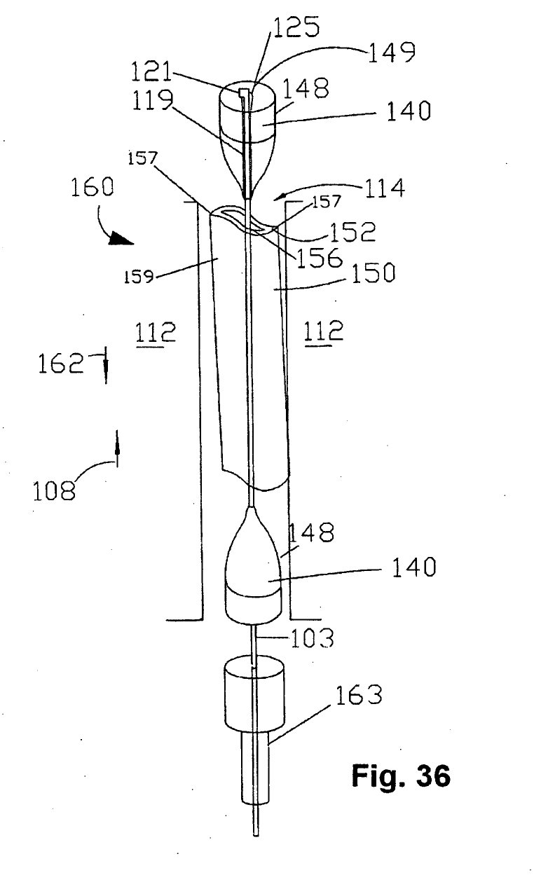

FIG. 36 shows a sheathed fastener 160 having two shafts 140; and mounted on guide wire 103. Tool 163 is pushed in proximal direction 108 while guide wire 103 is pulled in distal direction 162, thereby causing two shafts 140 to move toward each other within sheath 150.

Tool 163 is optionally configured to press against pre-deployed sheath 150 to aid in aligning two shafts 140 within sheath 150. Following movement of sheath 150, guide wire 103 is turned, so the projection aligns with channel 119, and is removed, as noted above.

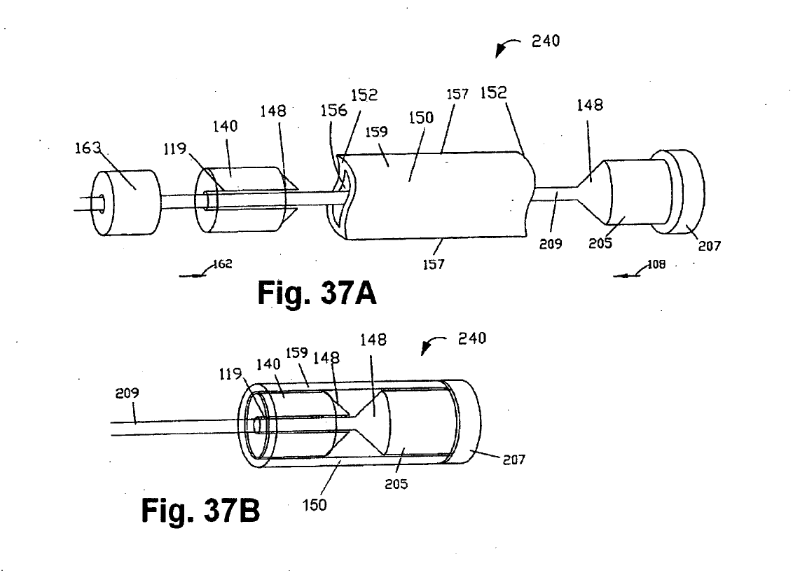

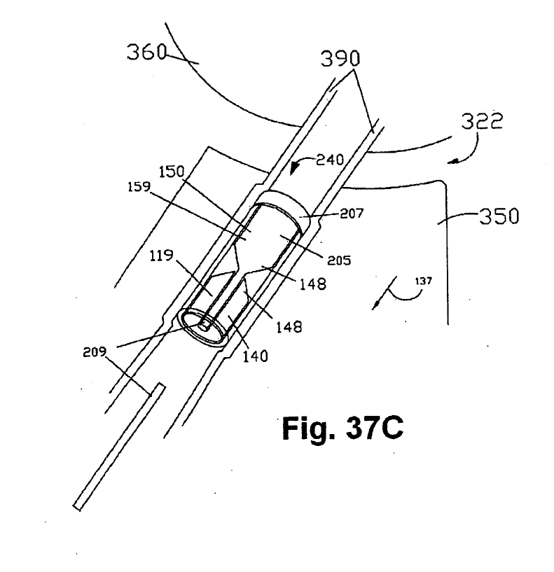

FIG. 37A shows an integrated guide fastener 240 having an elongate guide member 209 projection from a shaft 205 and passing through guide wire channel 119 in shaft 140.

During deployment, tool 163 is pressed against shaft 140 in direction 162 while guide member 209 is pulled in direction 108, causing shafts 140 and 205 to enter and expand sheath 150. A radial ledge 207 presses into cam edge 152, causing shaft 205 to enter sheath 150.

FIG. 37B shows integrated guide fastener 240 with flat shaft 150 in a deployed configuration. FIG. 37C shows integrated guide fastener 240 deployed in knee joint 322 with elongate guide member 209 projecting from a shaft 205 having been severed from fastener 240 using any one of several types of cutting tools.

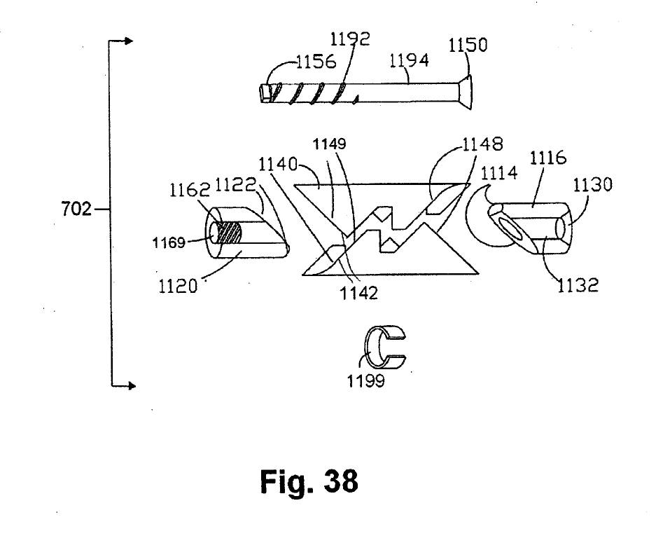

FIG. 38 shows a dual wall fastener 702 that includes a shaft 1194 having a threaded portion 1192, a square end 1156, and a flare end 1150. Walls 1149 include forward oblique surfaces 1142 and rearward oblique surfaces 1148.

A threaded securing nut 1120 having a bore 1169 with threads 1162 and includes bevels 1122 that interface with forward oblique surfaces 1142. A flare nut 1116 includes a bore 1132 that encircles shaft 1194 and a flare cavity 1130 that encircles flare end 1150. Beveled surfaces 1114 interface with rearward oblique surfaces 1148.

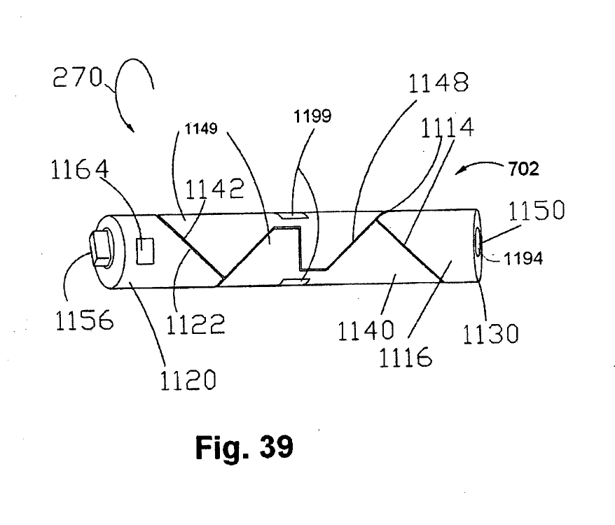

FIG. 39 shows assembled dual wall fastener 702, in which threaded securing nut 1120 includes grasping flats 1164 for grasping with a stabilizing instrument (not shown).

To cause radial outward movement, square end 1156 is rotated in a direction 270. By grasping flats 1164, threaded nut 1120 is stabilized against rotating and prevents walls 1149 and flare nut 1116 from rotating.

Shaft 1194 rotates in direction 270, causing threaded securing nut 1120 to move linearly toward flared nut 1116. In an exemplary embodiment, nut 116 remains stationary, while flare end 1150 rotates.

In the pre-deployed configuration, the side edges of portions of walls 1149 are located between each other, herein interdigitation of side edges, to aid in maximizing the diameter of dual wall fastener 702 that is in contact with tissue in the radial outward position.

The inventor has discovered that in addition to maximizing bone contact, graft strands 390 (FIG. 37C), may possibly be more likely pressed against bone 350, rather than being positioned between walls 1149; as may be the case with the teachings of PCT Application US2007/006928 (Montgomery et al).

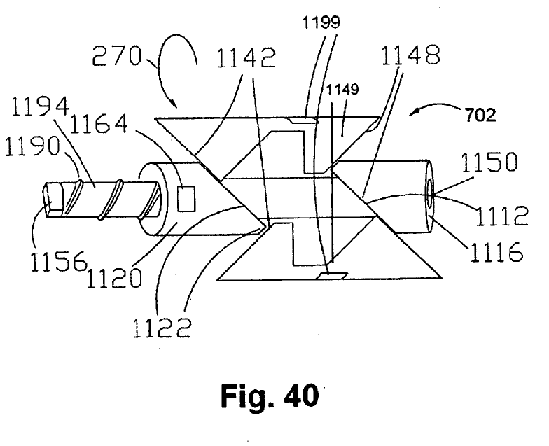

FIG. 40 shows dual wall fastener 702 with walls 1149 in a radially outward position as driven by securing nut 1120 moving linearly toward flared nut 1116.

A partial ring 1199 of a flexible hard material, for example surgical spring steel, provides radial inward pressure against walls 1149 to maintain proximity to shaft 1194.

In an alternative embodiment, a ring (not shown) that completely surrounds dual wall fastener 702, for example manufactured from an elastomeric material, may be used in place of partial ring 1199.

### Femoral Prosthetic Stem

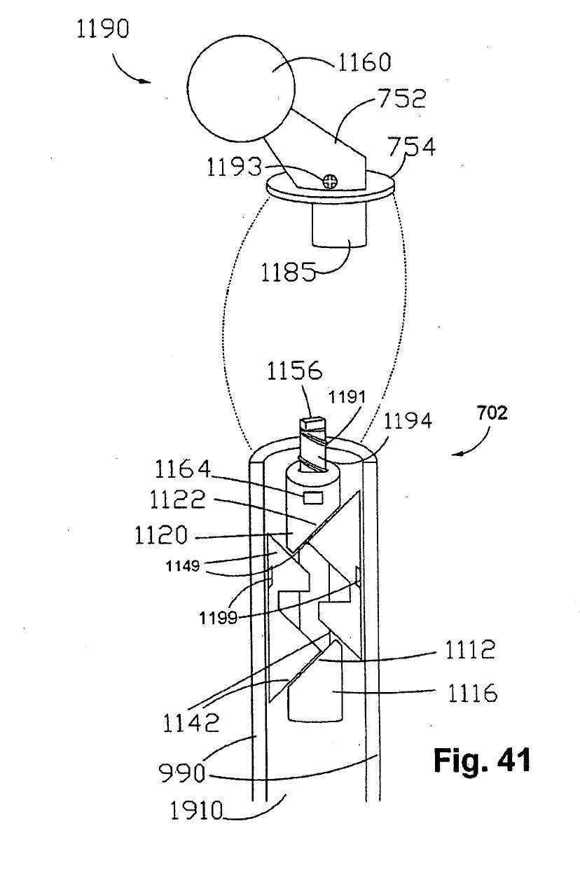

FIG. 41 shows dual wall fastener 702 installed in a cross section of an upper femur 1910 and femoral head 1190 being attached to shaft 1194. A prosthetic base 1185, for example, has inner threads that screw onto threads 1191. A securing screw 1193 is optionally provided and tightened to maintain alignment of prosthetic head 1160. The large surface area of compression surfaces of walls 1149 serve to spread forces and substantially prevent resorption of cortex 990.

### Slide Nut

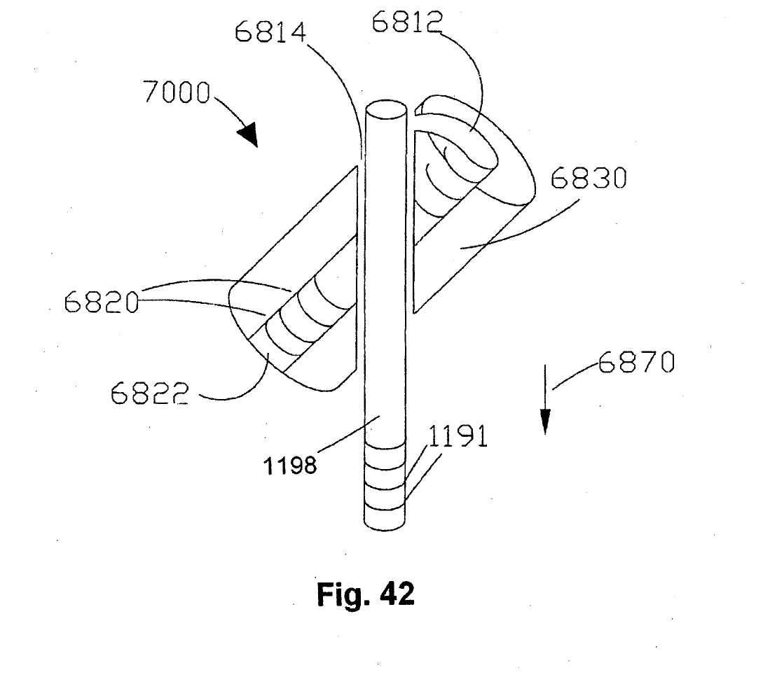

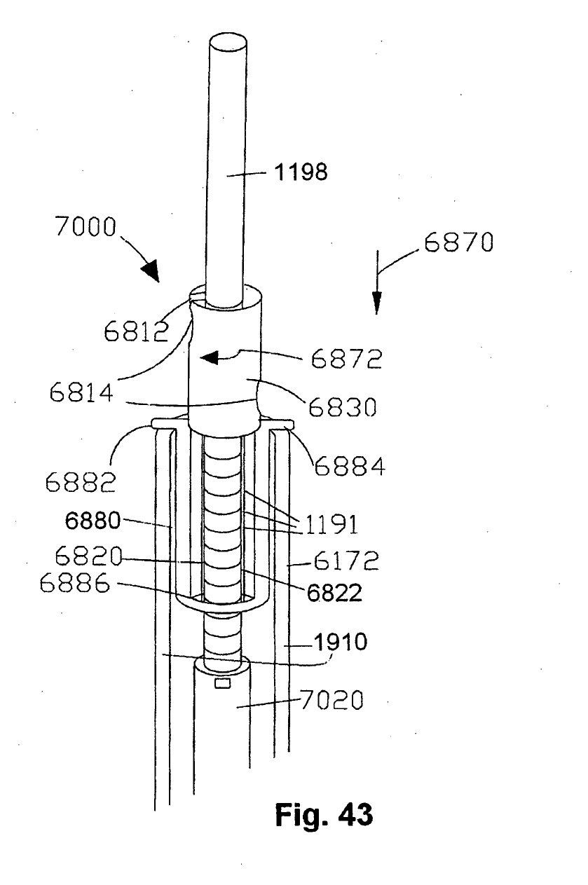

FIGS. 42-43 show the operation of a slide nut 7000 that secures to threaded shaft 1198. Slide nut 7000 includes a body 6830 having a non-threaded passage 6814 whose diameter is larger than threaded shaft 1198 and a threaded passage 6822 having threads 6820 adapted to interface with shaft threads 1191. Threaded passage 6822 is connected to non-threaded passage 6814 by a tilt passage 6812.

In FIG. 42, shaft 1198 is aligned with non-threaded passage 6814 so that nut 7000 slides linearly along shaft 1198 in a direction 6870 without rotating nut body 6830.

In FIG. 43, nut body 6830 has been pivoted so that shaft 1198 is passes through tilt passage 6812 and aligns with threaded passage 6822 and shaft threads 1191 interface with nut body threads 6820. Movement of nut body 6830 in direction 6870 occurs by rotation in a direction 6872. Nut body 6830 is rotated until body 6830 is substantially surrounded within a stabilizing cup 6880.

Stabilizing cup 6880 includes tabs 6882 and 6884 that secure to bone 1910. Optionally, cup 6880 includes a friction surface 6886 that interfaces with nut body 6830. Rotation counter to direction 6872 allows nut body 6830 to loosen.

### Femoral Condyle Fixation

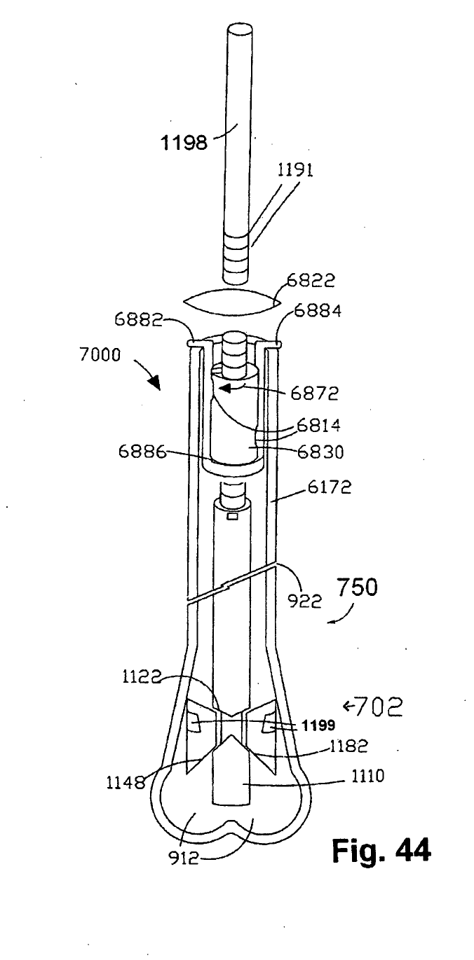

FIG. 44 shows a dual wall fastener 750 installed in a cross section of a femur 6172 having a fracture 922 near femoral condyles 912.

Nut 7000 provides compression force along fracture 922 that is adjusted by rotating nut body 6830 in direction 6872 or counter to direction 6872. Typically, shaft 1198 is cut flush with nut body 6830 and removed through a small incision 6822 that was used for insertion of dual wall fastener 702 and nut 7000.

### Spiral Wall Fastener

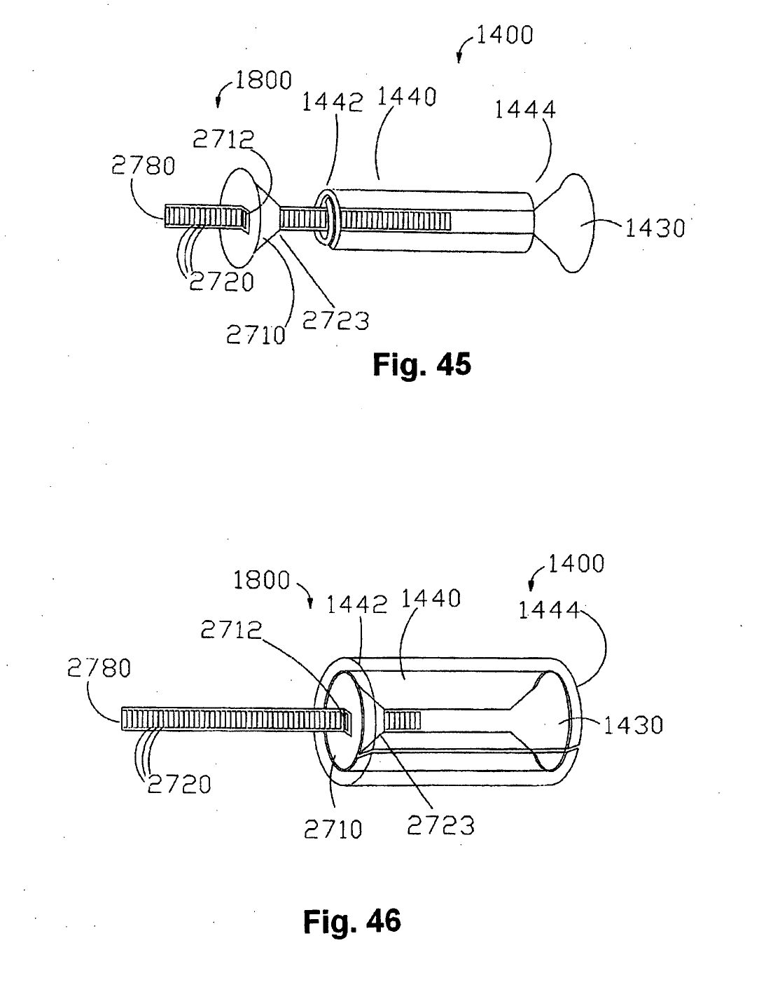

FIG. 45 shows a spiral wall fastener 1400 having a ratchet shaft 2780 with ratchets 2720. A ratchet securing mechanism 1800 includes a ratchet interface 2712, secures on ratchets 2720 and includes a rearward flare 1430, and a spiral wall 1440.

As a ratchet nut 2710 moves toward flare 1430, a forward spiral wall edge 1442 is pressed by a forward nut surface 2723 and rearward spiral wall edge 1444 is pressed by flare 1430, causing spiral wall 1440 to move radially outward, until, as seen in FIG. 46, spiral wall 1440 is at a maximal diameter.

### Deployment Tool

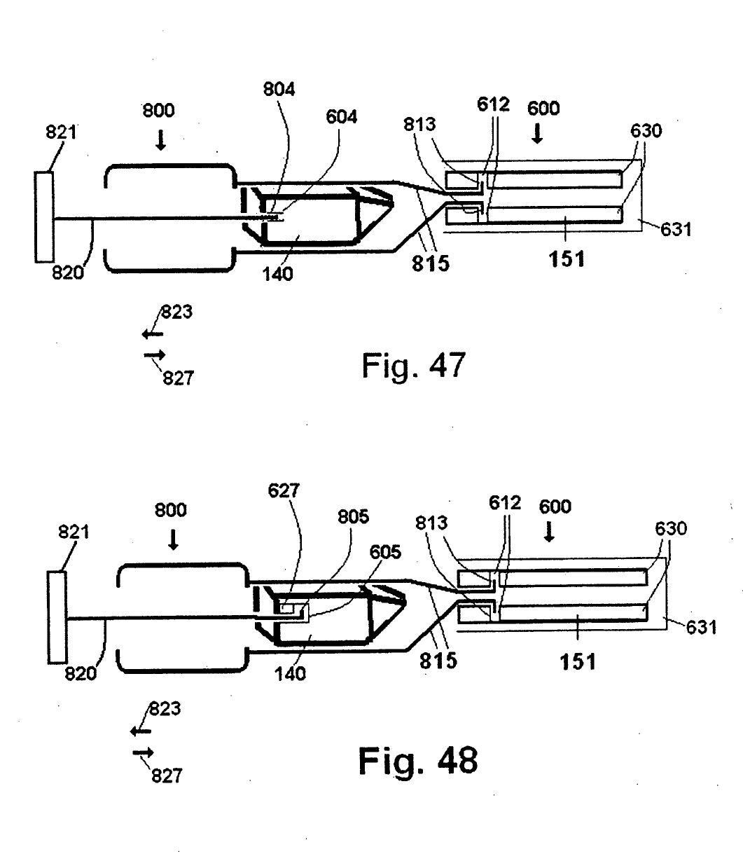

FIG. 47 shows a deployment tool 800 comprising two stabilizing posts 815 having stabilizing prongs 813, that interface with a fastener 600. Stabilizing prongs 813 at the ends of stabilizing posts 815 enter stabilizer ports 612 on jacket 151 of a fastener 600.

Additionally deployment tool 800 includes a shaft 820 having a threaded end 804 that threadingly affixes to a threaded receptacle 604 of shaft 140 on fastener 600.

Stabilizing prongs 813 at the ends of stabilizing posts 815 pass through shaft channels 612, while threaded end 804 is threaded into threaded receptacle 604.

It should be noted that fastener 600 could be any one of, or combination of, fasteners heretofore presented.

Deployment of fastener 600 is accomplished with a forward movement in a direction 827 of shaft 820 into a bone bore 631. A deployment plate 821 is pushed forward in direction 827 to cause shaft 140 to pass into jacket 151 and cause jacket 151 to expand radially outward. In embodiments, while jacket 151 expands, stabilizing posts 815 flex radially outward to maintain stabilizing prongs 813 in stabilizing ports 612.

To pull ram shaft 140 out of jacket 151 and collapse fastener 600, for example to reposition fastener 600, removal shaft 820 is pulled in a backward direction 823, so that shaft 140 is pulled out of jacket 151 and jacket 151 at least partially collapses. Fastener 600 may then be repositioned, after which shaft 140 is pushed into jacket 151 to secure fastener 600.

FIG. 48 shows an alternative embodiment of deployment tool 800 and fastener 600 in which deployment tool 800 includes a hook 805 that interfaces with a hook channel 605. Hook 805 is used to pull ram shaft 140 out of jacket 151 in the event that repositioning is desired.

To remove hook 805 from ram shaft 140, removal shaft 820 is rotated to align hook 805 with an exit channel 627. Removal shaft 820 is pulled in backward direction 823 to free hook 805 from shaft 140. Deployment tool 800 is then removed from dual wall fastener 600.

### Materials

In embodiments, sheath 150 and/or shaft 140 comprise a metallic base from the group consisting of: stainless steel, nitinol, tantalum, MP35N alloy, a cobalt-based alloy, a cobalt-chromium alloy, platinum, titanium, or other biocompatible metal alloys.

In embodiments, sheath 150 and/or shaft 140 comprise a bio degradable/bio-absorbable base from the group consisting of: PGLA, PLLA, PLA, bio-resorbable magnesium, or other bio resorbable compounds.

In embodiments, sheath 150 and/or shaft 140 comprise a material selected from the group consisting of: polyethylene, polyvinyl chloride, polyurethane, nylon and a biocompatible polymer fiber.

It is expected that during the life of a patent maturing from this application many relevant expanding fasteners will be developed and the scope of the term expanding fastener is intended to include all such new technologies a priori.

As used herein the term “about” refers to ±10%

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. An expanding fastener, comprising:
i) two walls configured to be inserted in a bore in a bone, said two walls including:
one first wall having at least one first flat planar wall portion; and
one second wall, and
ii) a shaft having one flat planar shaft portion configured to slide along said one first flat planar wall portion, said shaft further having a shape configured to cause radial outward movement of at least one of said two walls during said sliding.

2. The expanding fastener according to claim 1, wherein at least one of said two walls is configured to secure against at least a portion of said bore in said bone following said radially outward movement.

3. The expanding fastener according to claim 2, wherein said one first wall includes a first spine configured to secure a portion of graft, disposed in said bore, against a portion of said bore.

4. The expanding fastener according to claim 3, wherein said graft comprises at least one of:
a biocompatible material;
an autograft;
an allograft; and
a zenograft.

5. The expanding fastener according to claim 3, wherein said first spine includes at least one first rib having a free end extending toward said second wall.

6. The expanding fastener according to claim 5, wherein said at least one first rib is configured to press a portion of soft tissue disposed in said bore, against a portion of said bore.

7. The expanding fastener according to claim 3, wherein said one second wall includes a second spine.

8. The expanding fastener according to claim 7, wherein said second spine includes at least two ribs extending therefrom.

9. The expanding fastener according to claim 8, wherein said at least two ribs comprise:
at least one first rib extending from a first side of said second spine; and
at least one second rib extending from a second side of said second spine.

10. The expanding fastener according to claim 3, wherein prior to said radially outward movement at least a portion of said one flat planar shaft surface is parallel to said at least one first flat planar wall.

11. The expanding fastener according to claim 3, wherein following said radial outward movement at least a portion of said one flat planar shaft surface is parallel to said at least one first flat planar wall.

12. The expanding fastener according to claim 3, wherein following said radial outward movement at least a portion of said one first flat planar wall portion is parallel to said one flat planar shaft portion.

13. The expanding fastener according to claim 1, wherein said one first wall comprises at least one fold substantially along a longitudinal axis of said fastener.

14. The expanding fastener according to claim 1, wherein at least a portion of:
said one first wall; and
said one second wall,
are substantially radially continuous around said shaft.

15. The expanding fastener according to claim 1, including an elongate stabilizing cord projecting from said one first wall, said cord configured to stabilize the position of said one first wall, during said radial outward movement.

16. The expanding fastener according to claim 15, including an elongate shaft cord projecting from said shaft, said cord being configured to pull said shaft during said radial outward movement.

17. A method for compressing an in vivo tissue, the method comprising:
a) boring a bore through a surface of an in vivo tissue;
b) positioning at least two walls in said bore;
c) linearly sliding a flat shaft between said at least two walls; and
d) causing at least a portion of at least one of said at least two walls to move laterally outward and press against at least a portion of said bore.

18. A fastening device, comprising:
a first spine spaced a distance from a second spine:
said first spine having at least one first transverse rib extending toward said second spine,
said second spine having at least one second transverse rib extending toward said first spine and spaced a distance from said at least one first rib; and
an elongate insertion member insertable into said space between said first spine and said second spine, thereby expanding said first and second spines laterally outwardly.
